(19) United States
(12) Patent Application Publication
(10) Pub. No.: US 2008/0249484 A1
(43) Pub. Date: Oct. 9, 2008

(54) Auxiliary Probes

(75) Inventor: Andrea Venturelli, Concesio (Brescia) (IT)

Correspondence Address: SHOEMAKER AND MATTARE, LTD, 10 POST OFFICE ROAD - SUITE 110, SILVER SPRING, MD, 20910, US

(73) Assignee: INVATEC S.r.l., Roncadelle (Brescia) (IT)

(21) Appl. No.: 12/067,948

(22) PCT Filed: Oct. 28, 2005
(86) PCT No.: PCT/IT2005/000633

§ 371 Date: Apr. 22, 2008

(51) Int. Cl. A61M 25/14

(52) U.S. Cl. 604/284

(57) ABSTRACT

A first aspect of the invention relates to an auxiliary catheter comprising a main body comprising, in turn, a first duct defining a first lumen and a second duct defining a second lumen. Along the main body from the distal end in the proximal direction, the followings are found in the order:
the distal end of the first duct,
the side opening of the first duct, and
the distal end of the second duct comprising the front opening.

A second aspect of the invention relates to a kit comprising an equipped catheter and an auxiliary catheter having the same size as the equipped catheter. Finally, the invention relates to the methods for using these devices.

The objects of the present invention are auxiliary probes, particularly auxiliary catheters for endoluminal operations.

In the field of interventions in closed-ducts, such as pipelines or the like, it is known to use devices, generally defined as probes, which are adapted for being inserted and for acting, by means of an accessorized end, on those anomaly conditions that are found within the ducts.

In the medical field, in the particular field of operations on patients by the endoluminal route, it is known to use devices adapted to be inserted into either the blood vessels or other natural ducts of the human body. These devices, generally defined as catheters, are usually provided with an end equipped for acting on those pathological conditions found either within vessels or ducts of the human body. These catheters allow the operator to reach the disease site by the suitably equipped end, and hence to act on the latter.

In order to reach the site within the duct affected by the anomaly, the use of a guide wire is commonly known.

In the medical field, the guide wire is inserted into a large-sized vessel by means of a cutaneous incision, and it is then advanced along the vessel towards the disease.

The disease is often located in a secondary vessel, away from the vessel which is reached by means of the cutaneous incision. In this case, a tortuous path towards the disease is imposed to the guide wire because of frequent changes of direction, and a number of bifurcations are present in which the operator has to be able to take the proper vessel.

The operator follows, in a known manner, the path of the guide wire by means of angiographic techniques highlighting the configuration of the blood vessels and the position of the guide wire, by introducing a contrast liquid and using suitable radiographic equipment.

At the end of this long operation, a catheter may be caused to slide along the guide wire and to automatically reach the disease site.

From what has been stated above, it is understood that most of the time spent in order to reach the site to be treated is devoted to the introduction of the guide wire, whereas the introduction of the probe or catheter is a decidedly quicker operation.

A similar statement can be made for the error possibilities connected with the operation. The guide wire introduction step entails a constant risk of error, especially in terms of defining the exact, path in order to reach the anomaly to be treated.

For the reasons described above, the guide wire introduction step, though being only introductory to the downright operation, is somewhat critical with regard to the time and care required from the operator.

The introduction of an individual guide wire along which a single catheter is caused to slide is sufficient for a simple operation and during which no unforeseen event occurs, for example a PTCA (Percutaneous Transluminal Coronary Angioplasty) operation without any, complications.

On the other hand, when the type of operation is more complicated, for example just because the disease is located at a vessel, bifurcation, or because a combination of actions to be carried out with different catheters is required, then the single guide wire becomes immediately insufficient.

However the introduction of two or more guide wires by the conventional method entails a considerable series of drawbacks.

First of all, the introduction of each of the guide wires requires the same attention in terms of time and care being taken from the operator. In other words, the overall technical times, and error possibility to which the operator is exposed directly depend on the number of guide wires employed.

Then, the different guide wires are very likely to twine or twist around each other, along the tortuous path from the cutaneous incision to the disease site. In the event that this may happen, it is extremely difficult for the operator to be able to introduce the respective catheters.

In the technique of catheters for endoluminal operations, two different technical solutions are known relative to the manner in which the ducts intended to slide along the guide wire are manufactured.

A first solution commonly called over-the-wire provides that the duct of the catheter intended to accommodate the guide wire and to slide along the latter, has the same length as the whole catheter. In other words, in a catheter of the over-the-wire-type, the duct for the guide wire is provided with a distal port or opening near the catheter distal end, and a proximal port near the catheter proximal end.

Historically, this first solution is the first which has been employed and allows the catheter to be excellently supported by the wire.

A second solution commonly called rapid-exchange provides that the duct of the catheter intended to accommodate the guide wire and to slide along the latter, is decidedly shorter than the whole catheter. In other words, in a catheter of the rapid-exchange-type, the duct for the guide wire is provided with a distal port near the catheter distal end, and a proximal opening, being usually set in the catheter section immediately proximal to the equipped end.

This second solution allows a quicker disengagement of the catheter from the guide wire.

In the technique of the endoluminal operations, it is common to use catheters having highly sophisticated tools and technical solutions. These catheters are strictly of the disposable type, for understandable sanitary reasons; therefore, they are destined to be disposed of after they have been used.

The catheters for endoluminal operations are generally in different sizes, to be selected based on the size of the vessel to be treated and the tortuosity of the path that the catheter has to cover in order to reach the site to be treated.

In other words, the operator estimates the size of the vessel and the possible difficulties of the path to be covered in order to reach the disease. The operator selects the size of the most suitable catheter, based on these estimates.

It should be noted on this subject that, generally, a too small catheter is not capable of acting on the disease as effectively as possible, whereas a too big catheter is not even capable of reaching the site.

The prior art provides that the operator immediately proceeds with the introduction of the catheter equipped for the endoluminal operation, in consequence of the estimate carried out.

In any case, this prior art is not without drawbacks. For example, if the estimate carried out by the operator is either vitiated or is inaccurately carried out for any reason, a catheter which has been inserted and which is too big, has to be extracted and removed. Therefore, this event means a large waste because it makes the disposal of the equipped catheter necessary. In fact, in equipped catheter, though being substantially new because it has never had the function for which it has been manufactured and purchased, it has been in any case implanted into a patient and hence it has to be disposed of.

In the light of what has been stated above, in the field of devices for endoluminal operations the need for having a system and a device capable of solving the problems discussed with reference to the prior art is deeply felt.

The object of the present invention is to contrive and provide a probe and a method for using the latter, which are capable of solving the problems discussed with reference to the prior art.

Particularly, a task of the present invention is to provide an auxiliary catheter and a method for using, the latter in order to properly bring a plurality of guide wires to destination.

Another task of the present invention is to provide an auxiliary catheter and a method for using the latter, which allow a plurality of guide wires to be properly set in a relatively short time.

Another task of the present invention is also to provide an auxiliary catheter and a method for using, the latter, which allow positioning a plurality of guide wires while reducing the error possibilities to which the operator is exposed.

A further task of the present invention is to make a catheter and a method for using the latter available, which allow to directly check the size of a duct requiring the action, with no need to use an equipped catheter.

This object and these tasks are achieved by means of auxiliary catheters and kits it accordance with claims 1 and 16 and by means of respective methods for using the latter in accordance with claims 14, 15 and 20.

Further characteristics and the advantages of the catheter according to the invention will be understood from the description of preferred exemplary embodiments as set forth herein below, which are given by way of indicative and non-limiting example, with reference to the annexed figures, in which

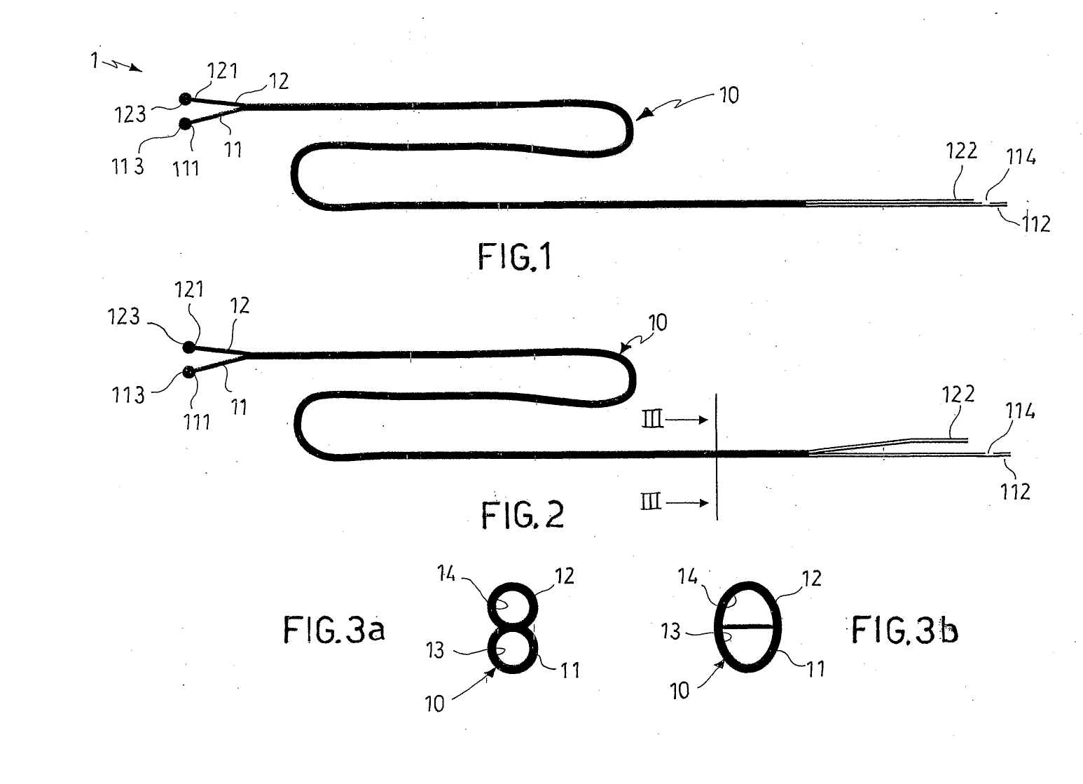

FIG. 1 shows an overall schematic view of an auxiliary catheter according to the invention;

FIG. 2 shows an overall schematic view of an auxiliary catheter according to the invention;

FIGS. 3.a and 3.b show two possible sections taken along plane III-III from FIG. 2;

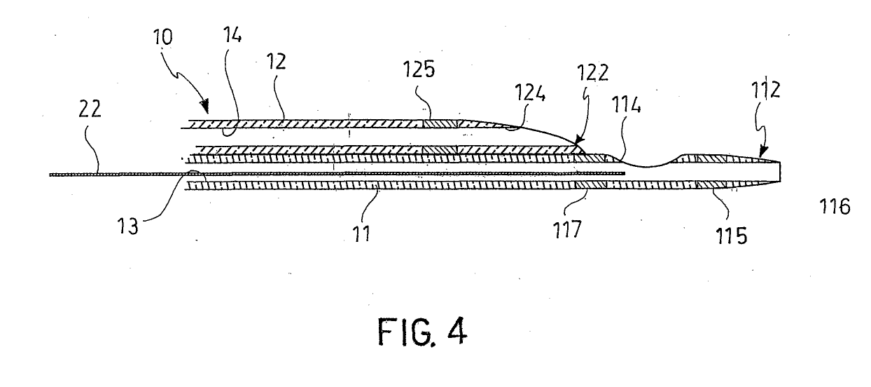

FIG. 4 shows a sectional view of the detail of the distal portion for a catheter similar to the one from FIG. 1;

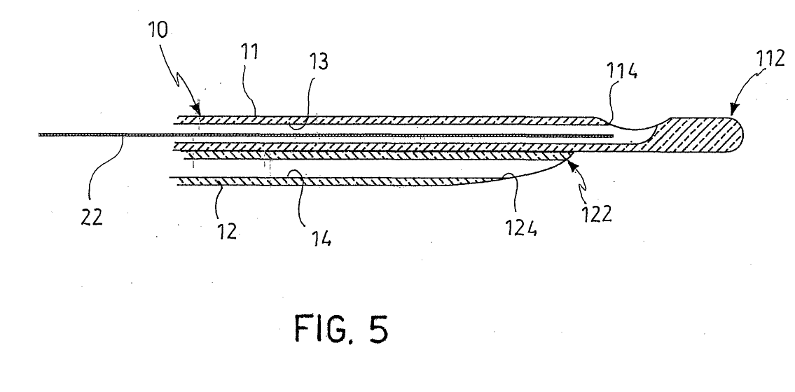

FIG. 5 shows a sectional view of the detail of the distal portion for a catheter according to the invention;

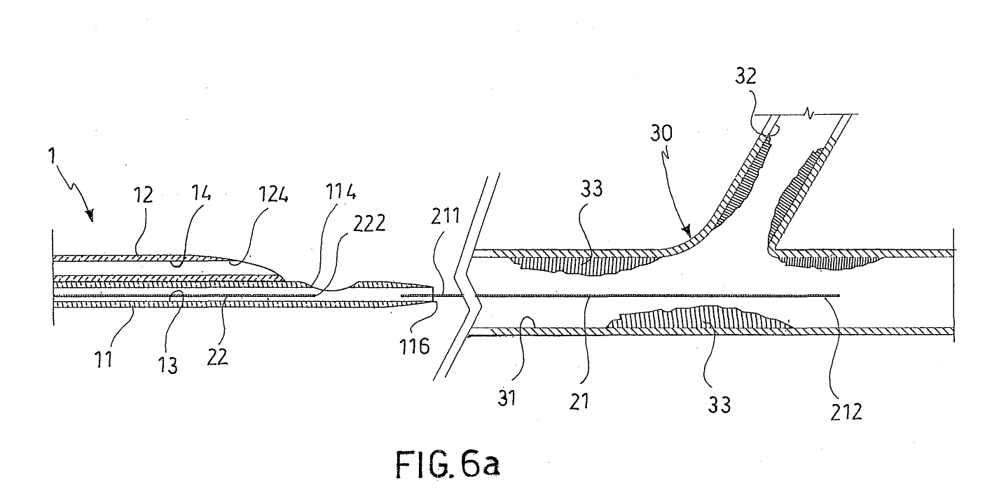

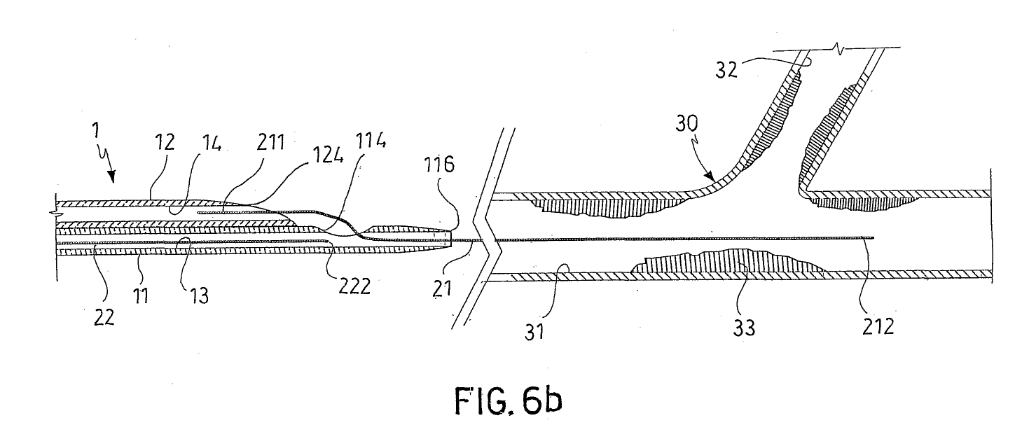

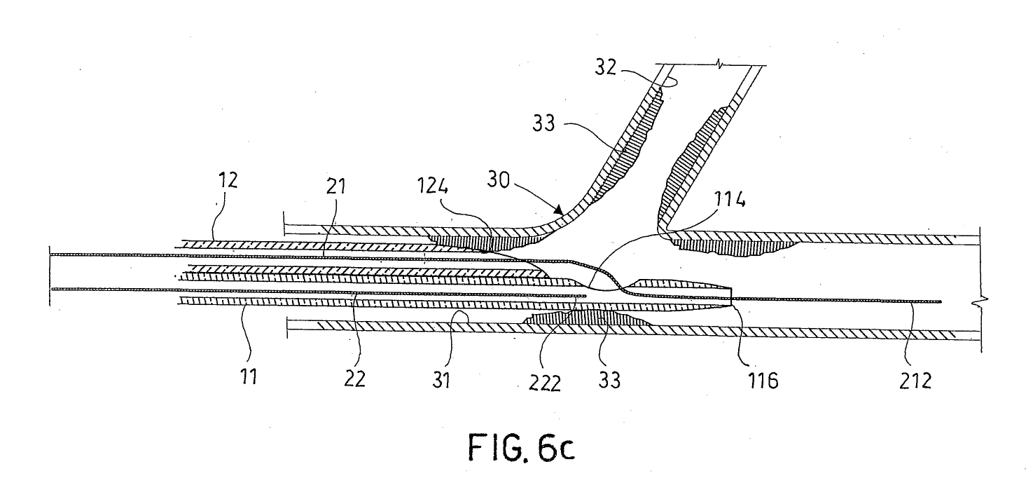

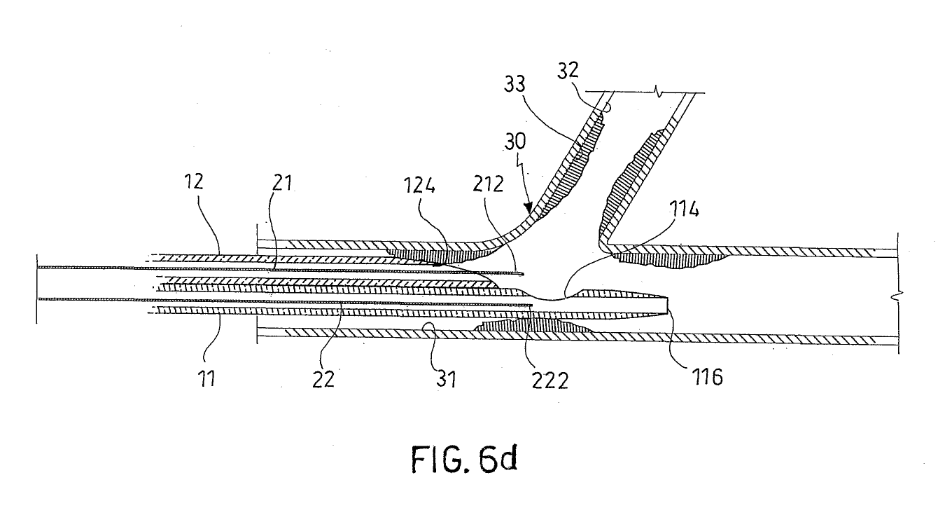

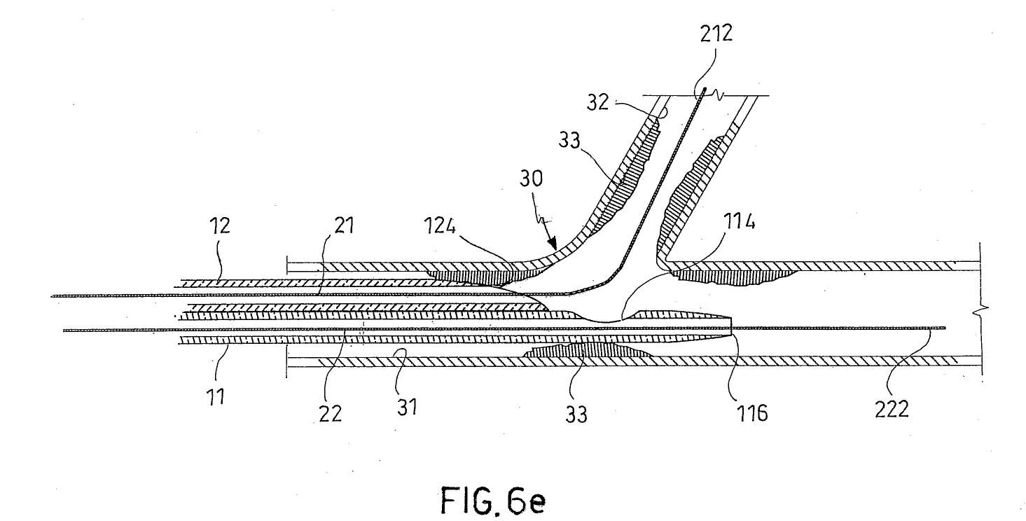

FIGS. 6.a to 6.e show some successive steps of the method for using the catheter from FIG. 4;

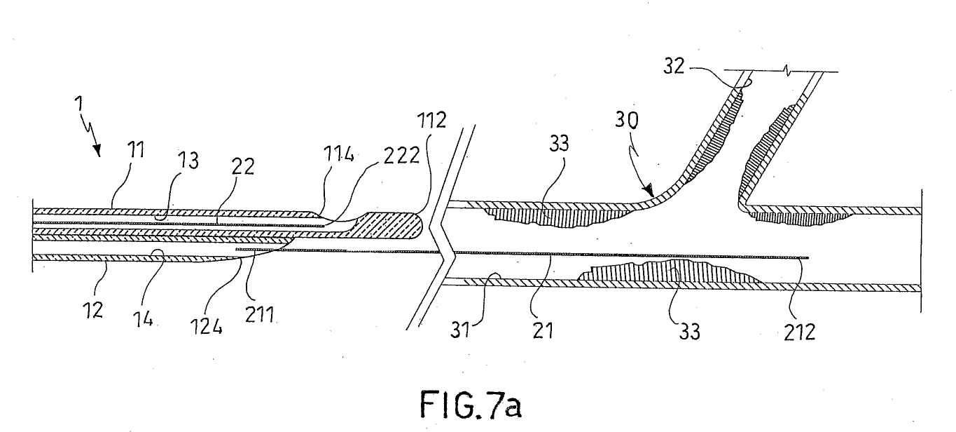

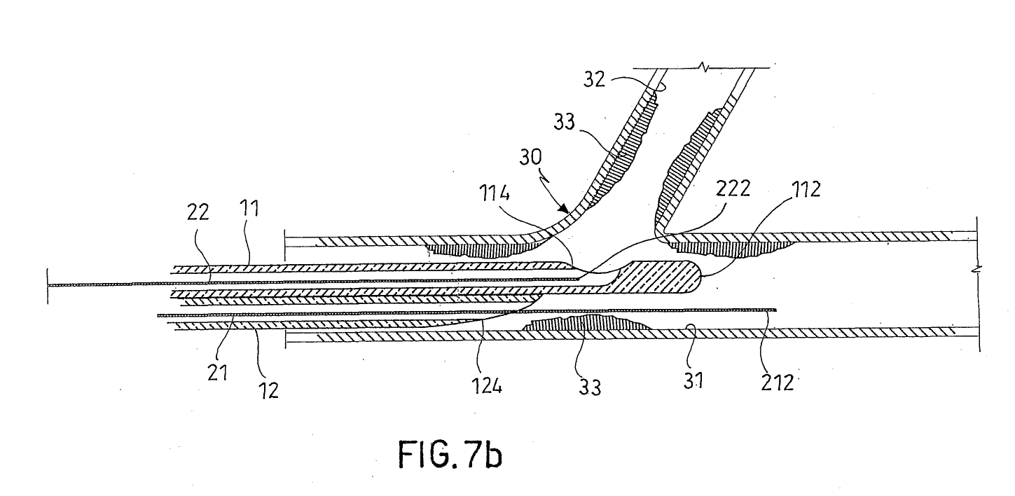

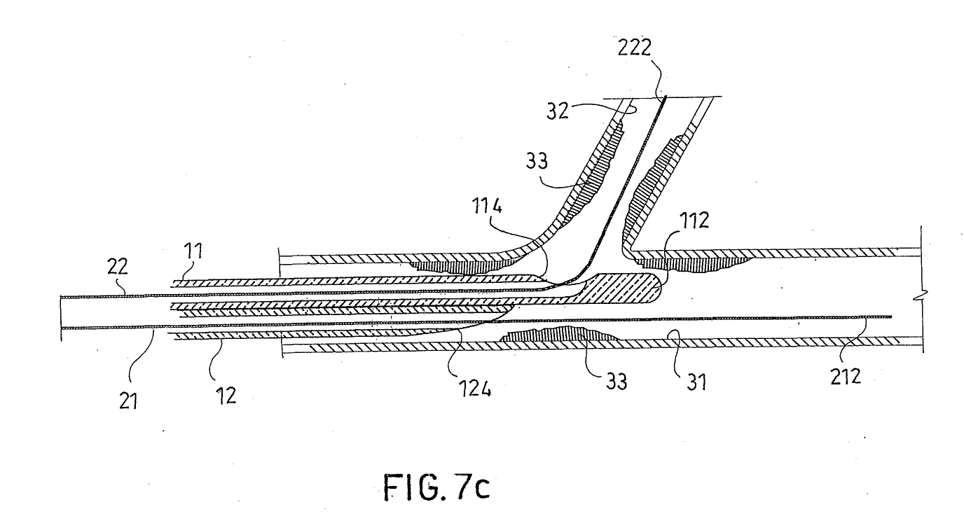

FIGS. 7.a to 7.c show some successive steps of the method for using the catheter from FIG. 5;

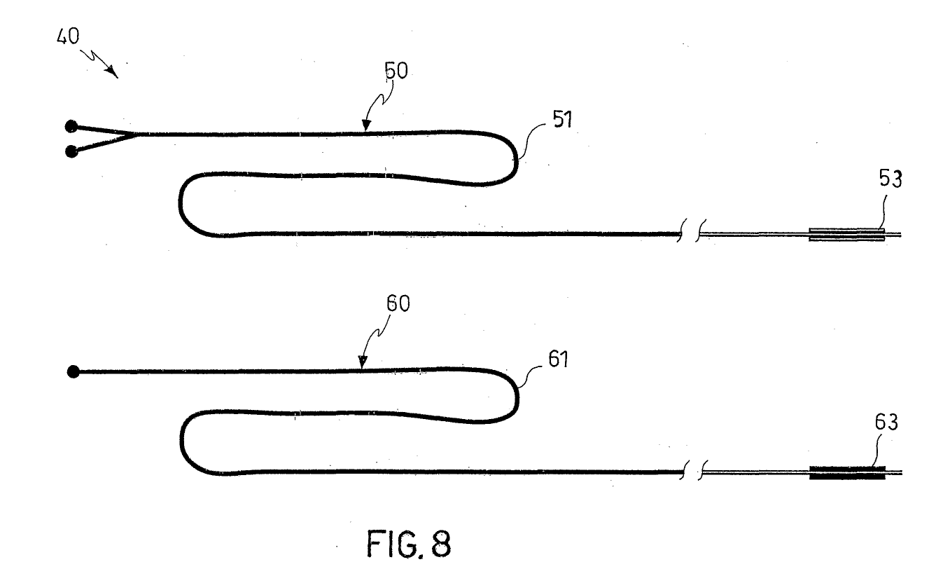

FIG. 8 shows an overall schematic view of a kit according to the invention;

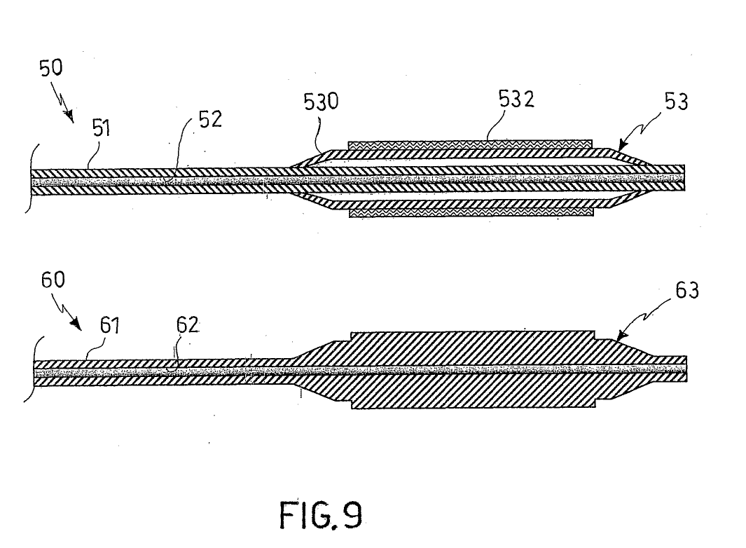

FIG. 9 shows a sectional view of the detail of the distal portions for a kit similar to the one from FIG. 8;

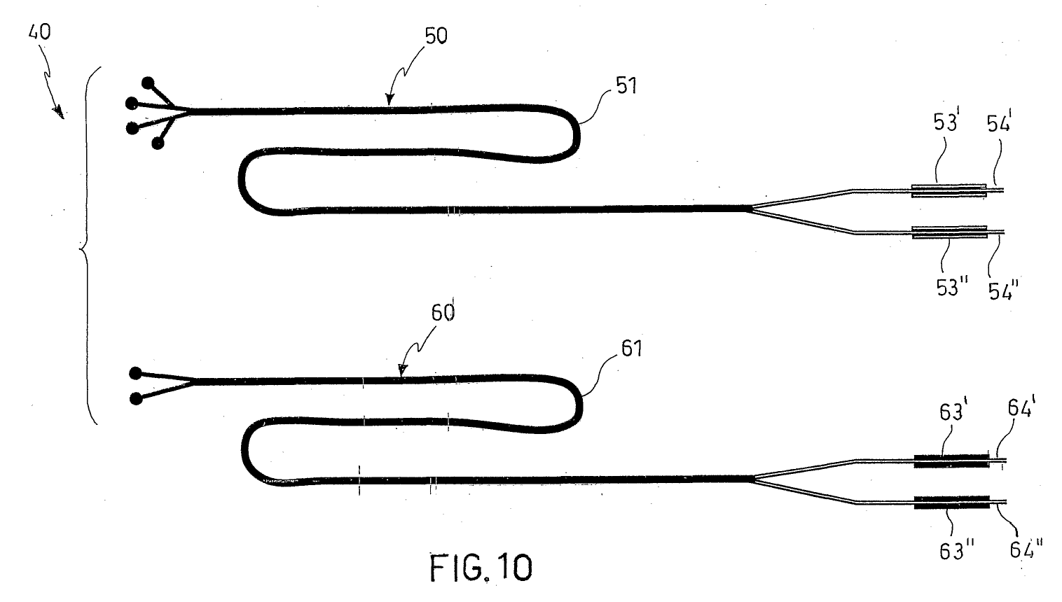

FIG. 10 shows an overall, schematic view of a kit according to the invention;

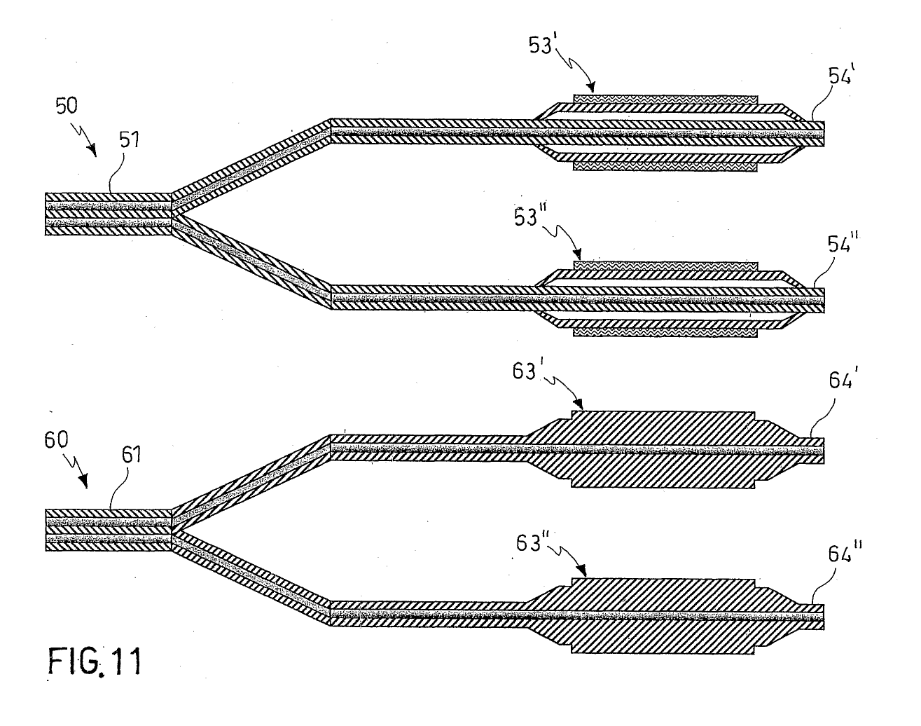

FIG. 11 shows a sectional view of the detail of the distal portions for a kit similar to the one from FIG. 10.

With reference to said figures, a first embodiment of an auxiliary probe according to the invention, particularly having the function of a catheter has been indicated with 1 as a whole.

The catheter 1 comprises a main body 10 being flexible and of an extended shape, provided with a distal end and a proximal end.

The distal end is intended to be inserted into a blood vessel or another natural duct of a living body upon use. On the contrary, the proximal end is intended to be outside the living body upon use.

The main body 10 comprises two ducts 11 and 12, each of which internally defines a lumen.

The first duct 11 defines a first lumen 13 extending from a proximal end 111 to a distal end 112. The first duct 11 comprises a known connector 113 at the proximal end 111 thereof, for example of the commonly called Luer-lock-type. The first duct 11 comprises a side opening 114, at the distal end 112.

The second duct 12 defining a second lumen 14 extending from a proximal end 121 to a distal end 122 is arranged side by side with the first duct 11. The second duct 12 also comprises a connector 123, at the proximal end 121 thereof, for example of the commonly called Luer-lock-type. The distal end 122 of the second duct 12 comprises a front opening 124.

As regards the elements described above and located at the distal end of the main body 10, they are set in a predetermined order, in accordance with the invention.

Along the main body 10 of the catheter 1 from the distal end in the proximal direction, the followings are found in the order:
the distal end 112 of the first duct 11,
the side opening 114 of the first duct 11, and
the distal end 122 of the second duct 12 comprising the front opening 124.

In accordance with an embodiment of the invention, for example such as illustrated in FIG. 4, the distal end 112 of the first duct 11 also comprises a front opening 116.

In accordance with an embodiment of the invention, for example the one illustrated in FIG. 1, 2 or 4, the side opening 114 of the first duct 11 opens on the same side of the first duct 11 with which the second duct 12 is placed side by side.

In accordance with another embodiment of the invention, for example such as illustrated in FIG. 5, the side opening 114 of the first duct 11 opens on the side of the first duct 11 which is opposite to the one with which the second duct 12 is placed side by side.

In accordance with another embodiment of the invention, for example the one illustrated in FIG. 4 or 5, the front opening 124 of the second duct 12 has an oblique shape. In other words, the distal end 122 of the second duct 12 is cut along a surface which is not perpendicular to the axis locally defined by the duct 12.

Particularly, as may be clearly seen in FIG. 4 or 5, if the distal end 122 is oblique, the latter should preferably have such a shape as to have a higher distal extension at the side of the second duct 12 with which the first duct 11 is placed side by side and as to have a lower distal extension at the side of the second duct 12 being opposite to the one with which the first duct 11 is placed side by side, respectively.

In accordance with a possible embodiment of the invention, the distal end 112 of the first duct 11 being the forward element in the distal direction of the whole main body 10, has a tapered shape. Particularly, the tapering may be obtained by providing the distal end 112 with an ogival, truncated cone or dome shape.

The presence of the optional front opening 116 and the tapering described above on the distal end 112, are substantially independent from each other.

In accordance with the embodiment from FIG. 3.a, the section of the main body 10, being carried out along a plane which is perpendicular to the axis locally defined by one of the two ducts, is of an ‘8’ shape. This shape is due to two simple one-lumen ducts being placed on the side, providing the main body 10.

In accordance with the embodiment from FIG. 3.b, the section of the main body 10 has a substantially circular shape and comprises two lumens being of a ‘D’ shape and approached along their respective flat sides. This shape is due to the use of a two-lumen duct providing the main body 10.

According to the embodiment shown in FIG. 2, the distal lengths of the first duct 11 and second duct 12 are separate from one another, so that the distal portion of the main body 10 takes a ‘Y’-forked shape.

In accordance with an embodiment of the auxiliary catheter according to the invention, the distal end of the main body 10 comprises at least one radiopaque marker.

The radiopaque marker may be for example of a ring shape or a band shape manufactured in a heavy metal (for example Tantalum, Gold, Platinum or Tungsten). Either the ring or the band may be wound around the distal end of the main body 10 in the catheter manufacturing steps.

As described above, the auxiliary catheter 1 according to the invention may be seen by means of the fluoroscopic techniques commonly employed during the endoluminal operations. Thereby, the operator is able to follow the position of the distal end of the catheter 1 along the patient's vessels throughout the endoluminal operation.

In accordance with an embodiment of the invention, the distal end of the catheter 1 comprises three radiopaque markers. The first of the three markers defined as ‘distal’ herein below and indicated with 115) is preferably distally set relative to the side opening 114 of the first duct 11. The second of the three markers defined as the ‘middle’ herein below and indicated with 117) is preferably set between the side opening 114 of the first duct 11 and the distal end 122 of the second duct 12. The third of the three markers (defined as the, ‘proximal’ herein below and indicated with 125) is preferably proximally set, relative to the distal end 122 of the second duct 12.

In accordance with this embodiment of the catheter 1, by covering the main body 10 of the catheter 1 from the distal end in the proximal direction, the followings are found in the order:
the distal end 112 of the first duct 11,
the distal marker 115;
the side opening 114 of the first duct 11,
the middle marker 117;
the distal end 122 of the second duct 12 comprising the front opening 124, and
the proximal marker 125.

This arrangement of the three markers 115, 117 and 125 allows the operator to better define the position of the catheter within the vessel and the position of the different openings relative to one another during the operation, by means of the usual radiographic technique.

Particularly, the arrangement of the three markers allows the operator to define whether the two ducts 11 and 12 either overlap or are side by side with each other and, particularly, in which direction the side opening 114 is oriented, relative to the lookout point of the radiographic apparatus.

It will be explained herein below the method of use of the auxiliary probe 1, according to the invention, for treating an anomaly 33 located in a bifurcation 30 of a duct system, with reference to FIGS. 6.a to 6.e.

The bifurcation 30 comprises a primary duct 31 and a side secondary duct 32.

The method of use of the auxiliary probe 1 according to the invention provides the following steps.

With reference to FIG. 6.a, arranging a first guide wire 21 along the primary duct 31 such as to set the distal end 212 of the first guide wire 21 near the bifurcation 30 to be treated, and such as to set the proximal end 211 of the first wire 21 so that the latter is outside the duct and easily reached by the operator.

Arranging a second guide wire 22 within the first lumen 13 of the probe 1 such as to set the distal end 222 of the second guide wire 22 near the side opening 114.

With reference to FIG. 6.b, inserting the proximal end 211 of the first wire 21 successively: into the front opening 116 so that it enters the first lumen 13, into the side opening 114 so that it comes outside the probe 1, and into the front opening 124 so that it enters the second lumen 14.

Causing the probe 1 to slide along the first guide wire 21 within the primary duct 31 such as to arrange the probe distal end near the bifurcation 30 to be treated, such as in FIG. 6.c.

Checking the orientation of the distal end of the probe 1 relative to the bifurcation 30 to be treated and, particularly, relative to the side secondary duct 32.

With reference to FIG. 6.d, withdrawing the first guide wire 21 along the second lumen 14 so that the distal end 212 of the first guide wire 21 passes through the front opening 116 such as to enter the first lumen 13, and the side opening 114 such as to come outside the probe 1.

With reference to FIG. 6.e, re-positioning the distal end 212 of the first guide wire 21 within the side secondary duct 32 of the bifurcation 30 to be treated

Causing the second guide wire 22 to advance along the first lumen 13 so that the distal end 222 of the second guide wire 22 passes through the front opening 116 such as to come outside the probe 1.

Extracting the auxiliary probe 1 thus leaving the guide wires 21 and 22 in position.

A particular embodiment of the method described above can be used for treating diseases imputed to a patient's circulatory system. In this specific case, the ducts to which reference is made for describing the method are the patient's blood vessels and the anomaly is, for example, a stenosis. In this embodiment, the probe to which reference is made for describing the method can be a catheter of the above-mentioned type, provided with a front opening 116 at the distal end of the first duct 11.

It will be described herein below, with reference to FIGS. 7.a to 7.c, another embodiment of the method according to the invention providing the following steps.

With reference to FIG. 7.a, arranging a first guide wire 21 along the primary duct 31 such as to set the distal end 212 of the first guide wire 21 near the bifurcation 30 to be treated, and such as to set the proximal end 211 of the first wire 21 so that it is outside the duct and easily reached by the operator.

Arranging a second guide wire 22 within the first lumen 13 of the probe 1 such as to set the distal end 222 of the second guide wire 22 near the side opening 114.

Inserting the proximal end 211 of the first wire 21 into the front opening 124 so that it enters the second lumen 14.

Causing the probe 1 to slide along the first guide wire 21 within the primary duct 31 such as to arrange the probe distal end near the bifurcation 30 to be treated, such as in FIG. 7.b.

Checking the orientation of the distal end of the probe 1 relative to the bifurcation 30 to be treated and, particularly, relative to the side secondary duct 32.

With reference to FIG. 7.c, causing the second guide wire 22 to slide along the first lumen 13 so that the distal end 222 of the second guide wire 22 passes through the side open ring 114 such as to come outside the probe 1.

Positioning the distal end 222 of the second guide wire 22 within the side secondary duct 32 of the bifurcation 30 to be treated.

Extracting the auxiliary probe 1 thus leaving the guide wires 21 and 22 in position.

A particular embodiment of the method described above can be used for treating pathologies imputed to a patient's circulatory system. In this specific case, the ducts to which reference is made for describing the method are the patient's blood vessels and the anomaly is, for example, a stenosis. In this embodiment, the probe to which reference is made for describing the method can be a catheter of the type of the one described above.

In the light of what has been stated above, it will be understood by those skilled in the art that the use of the catheter and the method according to the invention allow to overcome some serious drawbacks relative to the prior art.

As will be obviously appreciated by those skilled in the art, both the methods described above allow two guide wires 21 and 22 to be set in a bifurcation 30, by the use of an auxiliary probe 1, thus ensuring that they will not twine around each other.

Furthermore, only one of the two guide wires requires to be taken along the primary duct 31 up to the bifurcation 30 to be treated, whereas the other guide wire simply requires to be positioned within the proper lumen of the probe 1.

Without a shadow of doubt, the operation of causing the guide wire to cover the length being often tortuous taking the latter to the bifurcation 30 is the most complicated, difficult and longest one. The present invention allows to set two guide wires by carrying out this operation only once. For this reason, the invention allows the times and the risk possibilities connected with the operation to be reduced.

It is described herein below the kit 40 illustrated in FIGS. 8 to 11 and comprising a known equipped catheter 50 and an auxiliary catheter 60 according to the invention.

The equipped catheter 50 may be any catheter of the types commonly used for endoluminal operations, for example for PTCA (Percutaneous Transluminal Coronary Angioplasty) operations.

In accordance with an embodiment of the kit 40, the equipped catheter 50 comprises a tubular body 51 being flexible and of an extended shape, a duct 52 for a guide wire and acting means 53 in order to either diagnostically or therapeutically act on the site 30 to be treated. The auxiliary catheter 60 of the same kit 40 comprises a tubular body 61 being flexible and of an extended shape, a duct 62 for a guide wire and dummy means or form 63.

The kit 40 according to the invention is characterized in that the auxiliary catheter 60 exactly has the same size as the equipped catheter 50 when the latter is in the configuration which is adapted to be introduced into the patient's vessels. In other words, the auxiliary catheter 60 has the same shape and the same size (lengths, distances, diameters, etc.) of the equipped catheter 50, unless the manufacturing tolerance is provided.

Therefore, the kit 40 is provided starting from a traditional equipped catheter 50. The kit is then made complete by an auxiliary catheter 60 being provided such as to accurately reproduce the size of the equipped catheter 50.

In the example schematically shown in FIGS. 8 and 9, the case of an angioplasty and stenting catheter 50 is considered, in the suitable configuration so that it may be introduced into the patient's vessels.

In this case, the acting means 53 of the equipped catheter 50 comprise an angioplasty balloon 530, located in the distal section of the catheter 50, and a lumen (not shown) for inflating the balloon 530 covering the whole length of the catheter 50. An endoluminal prosthesis (or stent) 532 is arranged around the angioplasty balloon 530, in a collapsed configuration and wound around the tubular body 51. The stent 532 is also in a collapsed configuration.

In this case, as may be clearly seen in FIG. 9, the auxiliary catheter 60 comprises a form 63, located in the distal section, which accurately reproduces the size of the acting means 53 of the equipped catheter 50. Therefore, the form 63 has the same length and, particularly, the same diameters of the acting means 53.

In accordance with the embodiment of the kit 40 schematically shown in FIGS. 10 and 11, the equipped catheter 50 bifurcates into two separate distal ends 54′ and 54″, each of which supports its acting means 53′ and 53″.

In this case, the auxiliary catheter 60 also comprises a bifurcation and two separate distal ends 64′ and 64″, each of which supports a form 63′ and 63″ accurately reproducing the size of the acting means 53′ and 53″ of the equipped catheter 50. Therefore, the forms 63′ and 63″ have the same length and, particularly, the same diameters of the acting means 53′ and 53″.

The method for treating an anomaly 33 in a duct 31 using the kit 40 according to the invention, in accordance with one embodiment thereof, provides the following steps.

Arranging a guide wire 21 along the duct 31 such as to set the distal end 212 of the guide wire 21 near the anomaly 33 to be treated, and such as to set the proximal end 211 of the guide wire 21 so that the latter is outside the duct 31 and easily reached by the operator.

Causing the auxiliary catheter 60 to slide along the guide wire 21 within the duct 31 thus checking whether the form 63 of the auxiliary catheter 60 may be arranged near the anomaly 33 to be treated.

Extracting the auxiliary catheter 60 and leaving the guide wire 21 in position.

If the operation of arranging the form 63 of the auxiliary catheter 60 near the anomaly 33 has been successful, causing the equipped catheter 50 to slide along the guide wire 21 within the duct 31 such as to set the distal end of the equipped catheter 50 near the anomaly 33 to be treated.

If the operation of arranging the form 63 of the auxiliary catheter 60 near the anomaly 33 has not been successful, using a kit 40 of smaller sizes.

In the light of what has been stated above, it will be understood by those skilled in the art that the use of the kit 40 according to the invention allows to overcome some serious drawbacks relative to the prior art.

Particularly, the use of the kit 40 allows the operator to check the real possibility to reach the site to be treated with the equipped catheter 50 from a practical point of view, by means of the auxiliary catheter 60.

In fact, since the auxiliary catheter 60 is manufactured such as to exactly have the same size as the equipped catheter 50, the success in the attempt to reach the site to be treated with the auxiliary catheter 60 automatically means that the site can be also reached by means of the equipped catheter 50.

On the contrary, if the auxiliary catheter 60 is not capable of reaching the site to be treated, for example because of a restriction in the diameter of a vessel, it means that not even the equipped catheter 50 may reach the site.

Thereby, the equipped catheter 50 is kept in the sterile state thereof, and hence it can be used in a different operation.

To the embodiments of the auxiliary probes, the methods of use and the kits described above, those skilled in the art, in order to satisfy contingent needs, will be able to carry out modifications, adjustments and replacements of elements with other elements being functionally equivalent thereto.

For example, the catheter 1 can be indifferently provided either in the over-the-wire version or the rapid-exchange version, according to the specific needs.

Similarly, the equipped catheter 50 can be also indifferently provided either in the over-the-wire version or the rapid-exchange version, according to the specific needs. The auxiliary catheter 60 will be consequently provided either in the over-the-wire version or in the rapid-exchange version, exactly requiring the same size and the same behaviours of the equipped catheter 50.

Each of the characteristics described as belongings to a possible embodiment may be provided independently from the other described embodiments.

1-26. (canceled)

27. An auxiliary catheter comprising a main body comprising a first duct defining a first lumen and a second duct defining a second lumen, said ducts being placed side by side with each other, said first duct defining a side opening and said second duct defining a front opening, wherein, by covering said main body from the distal end in the proximal direction, the followings are found in the order:
the distal end of the first duct,
the side opening of the first duct, and
the distal end of the second duct comprising the front opening.

28. The auxiliary catheter according to claim 27, wherein the distal end of the first duct comprises a front opening.

29. The auxiliary catheter according to claim 27, wherein the side opening of the first duct opens on the same side of the first duct with which the second duct is placed side by side.

30. The auxiliary catheter according to claim 27, wherein the side opening of the first duct opens on the side of the first duct which is opposite to the one with which the second duct is placed side by side.

31. The auxiliary catheter according to claim 27, wherein the front opening and the distal end of the second duct have an oblique shape.

32. The auxiliary catheter according to claim 31, wherein the distal end has such a shape as to have a higher distal extension at the side of the second duct with which the first duct is placed side by side and as to have a lower distal extension at the side of the second duct which is opposite to the one with which the first duct is side by side, respectively.

33. The auxiliary catheter according to claim 27, wherein the distal end of the first duct has a tapered, ogival, truncated cone or dome shape.

34. The auxiliary catheter according to claim 27, wherein the cross section of the main body has an ‘8’ shape.

35. The auxiliary catheter according to claim 27, wherein the cross section of the main body has a substantially circular shape and comprises two lumens being of a ‘D’ shape and approached along their respective flat sides.

36. The auxiliary catheter according to claim 27, wherein the distal lengths of the first duct and second duct are separate from one another, so that the distal portion of the main body takes a ‘Y’-forked shape.

37. The auxiliary catheter according to claim 27, wherein the distal end of the main body comprises at least one radiopaque marker.

38. The auxiliary catheter according to claim 37, wherein the distal end of the main body comprises three radiopaque markers: a first distal marker distally set relative to the side opening of the first duct; a second middle marker set between the side opening of the first duct and the distal end of the second duct; and a third proximal marker proximally set relative to the distal end of the second duct.

39. The auxiliary catheter according to claim 27, wherein said first duct and said second duct are provided according to the over-the-wire solution.

40. The auxiliary catheter according to claim 27, wherein said first duct and said second duct are provided according to the rapid-exchange solution.

41. The auxiliary catheter according to claim 27, wherein said first duct is provided according to the over-the-wire solution and said second duct is provided according to the rapid-exchange solution.

42. The auxiliary catheter according to claim 27, wherein said first duct is provided according to the rapid-exchange solution and said second duct is provided according to the over-the-wire solution.

43. A method for using an auxiliary probe according to claim 27 for treating a bifurcation, comprising the steps of:
arranging a first guide wire along the primary duct such as to set the distal end of the first guide wire near the bifurcation to be treated, and such as to set the proximal end of the first wire so that the latter is outside the duct and easily reached by the operator;
arranging a second guide wire within the first lumen of the probe such as to set the distal end of the second guide wire near the side opening;
inserting the proximal end of the first wire into the front opening so that it enters the second lumen;
causing the probe to slide along the first guide wire within the primary duct such as to set the probe distal end near the bifurcation to be treated;
checking the orientation of the distal end of the probe relative to the bifurcation to be treated and, particularly, relative to the side secondary duct;
causing the second guide wire to slide along the first lumen so that the distal end of the second guide wire passes through the side opening such as to come outside the probe;
positioning the distal end of the second guide wire within the side secondary duct of the bifurcation to be treated;
extracting the auxiliary probe thus leaving the guide wires in position.

44. The method for using an auxiliary probe according to claim 28 for treating a bifurcation, comprising the steps of
arranging a first guide wire along the primary duct such as to set the distal end of the first guide wire near the bifurcation to be treated, and such as to set the proximal end of the first wire so that the latter is outside the duct and easily reached by the operator;
arranging a second guide wire within the first lumen of the probe such as to set the distal end of the second guide wire near the side opening;
inserting the proximal end of the first wire successively: into the front opening so that it enters the first lumen, into the side opening so that it comes outside the probe, and into the front opening so that it enters the second lumen;
causing the probe to slide along the first guide wire within the primary duct such as to set the probe distal end near the bifurcation to be treated;
checking the orientation of the distal end of the probe relative to the bifurcation to be treated and, particularly, relative to the side secondary duct;
withdrawing the first guide wire along the second lumen so that the distal end of the first guide wire passes through the front opening such as to enter the first lumen, and the side opening such as to come outside the probe;
repositioning the distal end of the first guide wire within the side secondary duct of the bifurcation to be treated;
causing the second guide wire to advance along the first lumen so that the distal end of the second guide wire passes through the front opening such as to come outside the probe;
extracting the auxiliary probe thus leaving the guide wires in position.

45. A kit comprising an equipped catheter comprising:
a tubular body,
a duct for a guide wire, and
acting means; and
an auxiliary catheter comprising:
a tubular body,
a duct for a guide wire, and
a form;
wherein the auxiliary catheter has the same size as the equipped catheter when the latter is in the configuration which is adapted for introduction into the patient's vessels, unless the manufacturing tolerance is provided.

46. The kit according to claim 45, wherein:
the acting means of the equipped catheter comprise at least one angioplasty balloon, and the form has the same size as the angioplasty balloon in the collapsed condition, unless the manufacturing tolerance is provided.

47. The kit according to claim 45, wherein:
the acting means of the equipped catheter comprise at least one balloon in a collapsed configuration and one endoluminal prosthesis, being also in a collapsed configuration, wound around the balloon, and
the form has the same size of the balloon in the collapsed condition and of the endoluminal prosthesis, being also in a collapsed configuration, wound around the balloon, unless the manufacturing tolerance is provided.

48. The kit according to claim 46, wherein
the equipped catheter bifurcates into two separate distal ends, each of which supports its acting means; and
the auxiliary catheter bifurcates into two separate distal ends, each of which supports a form in order to accurately reproduce the size of the acting means of the equipped catheter.

49. The kit according to claim 46, wherein said equipped catheter and said auxiliary catheter are both provided according to the over-the-wire solution.

50. The kit according to claim 46, wherein said equipped catheter and said auxiliary catheter are both provided according to the rapid-exchange solution.

51. A method for using a kit according to claim 46, comprising the steps of:
arranging a guide wire along the duct such as to set the distal end of the guide wire near the anomaly to be treated, and such as to set the proximal end of the guide wire so that the latter is outside the duct and easily reached by the operator;
causing the auxiliary catheter to slide along the guide wire within the duct thus checking whether the form of the auxiliary catheter may be arranged near the anomaly to be treated;
extracting the auxiliary catheter thus leaving the guide wire in position;
if the operation of arranging the form of the auxiliary catheter near the anomaly has been successful, causing the equipped catheter to slide along the guide wire within the duct such as to set the distal end of the equipped catheter near the anomaly to be treated;
if the operation of arranging the form of the auxiliary catheter near the anomaly has not been successful, using a kit of smaller sizes.
